(19) United States
(12) Patent Application Publication
(10) Pub. No.: US 2016/0000616 A1
(43) Pub. Date: Jan. 7, 2016

(54) Self-Cohesive Tape

(71) Applicant: David Michael Lavoie, Peterborough, NH (US)

(72) Inventor: David Michael Lavoie, Peterborough, NH (US)

(21) Appl. No.: 14/731,703

(22) Filed: Jun. 5, 2015

Related U.S. Application Data
(60) Provisional application No. 62/020,648, filed on Jul. 3, 2014.

(51) Int. Cl. A61F 15/00; A61F 13/02

(52) U.S. Cl. CPC A61F 15/005; A61F 13/0253; A61F 13/0206; A61F 13/0269

(57) ABSTRACT

A bandage wrapping system provided by the present invention includes an elongated strip of cohesive material and a dressing pad. The elongated strip of cohesive material includes a self-cohesive interior surface and exterior surface which are secured in a loop by overlapping opposing ends of the exterior layer and the interior layer. The dressing pad is configured to move along a length of the interior surface of the elongated strip of cohesive material and bonds thereto. The dressing pad adheres to the interior surface of the elongated strip via a layer of cohesive bonding material. The bandage wrapping conforms to any area by stretching and contracting with the movement of that area to stay in place.

### CROSS REFERENCE TO RELATED APPLICATION

This application claims the benefit of U.S. Provisional Application No. 62/020,648 filed on Jul. 3, 2014. The above identified patent application is herein incorporated by reference in its entirety to provide continuity of disclosure.

### FIELD OF THE INVENTION

The invention generally relates to bandages and wound dressings. More specifically, the present invention relates to a self-cohesive bandage with a mobile dressing pad.

### BACKGROUND OF THE INVENTION

Many individuals employ the use of bandages in order to cover an open wound, cut, or scrape to prevent the wound from becoming infected or further injured. Traditional bandages often contain sticky adhesives that become bonded to the skin and can cause pain when removed therefrom. This can be especially problematic for children who are at a higher risk of cuts and scrapes, and are particularly sensitive to pain from removing traditional bandages. Further, most bandages having adhesives can slip off over time and in instances when the skin is wet or oily. This can be frustrating and ineffective. Thus, there exists a need for a bandage that can be applied to wet or oily skin without the use of sticky adhesives that will remain on the skin for prolonged periods of time.

One ubiquitous type of adhesive bandage includes a strip of material, typically plastic or cloth, with a pressure sensitive adhesive on one side. A gauze pad is disposed on the same side as the adhesive, and a release liner covers the adhesive side of the strip material and the gauze pad. The entire bandage is enclosed in a package that maintains the sterility of the bandage until it is opened by the user. The bandage is then applied by removing the release liner, positioning the gauze pad over a wound, and adhering the adhesive side of the strip to the skin.

Although this type of adhesive bandage is effective for protecting cuts and scrapes from additional damage and bacteria, it does not solve the above identified problems. The adhesive bandage does not provide adequate protection for uneven surfaces, such as digits, and is susceptible to falling off the skin when the skin is wet or oily. Thus, there is a need for a wound dressing that provides adequate adhesion for wet or oily skin on hard to reach areas.

### SUMMARY OF THE INVENTION

In view of the foregoing disadvantages inherent in the known types of wound dressings now present in the prior art, the present invention provides a bandage wrapping system having an elongated strip of cohesive material with a movable dressing pad wherein the same can be utilized for providing convenience for the user when covering a wound on a digit or a limb.

It is therefore an object of the present invention to provide a new and improved bandage wrapping system that has all of the advantages of the prior art and none of the disadvantages.

It is another object of the present invention to provide a bandage wrapping system adapted to cover a wound on a digit or a limb. The bandage wrapping system includes an elongated strip of cohesive material having an interior surface and an exterior surface. The interior surface and the exterior surface comprise a self-cohesive layer. The bandage wrapping system conforms to any area by stretching and contracting with the movement of that area to stay in place and flush against the skin.

It is yet another object of the present invention to provide a dressing pad configured to be removably positioned along a length of the interior surface of the elongated strip of cohesive material. The dressing pad includes a layer of cohesive bonding material on a backside thereof for adhering to the elongated strip of cohesive material. The dressing pad adheres to the interior surface of the elongated strip of cohesive material via the layer of cohesive bonding material. The dressing pad includes a sterile non-stick pad.

Another aspect of the present invention is to provide a dispensing reel for elongated strip of cohesive material. The dispensing reel includes a snap holder for securing the elongated strip of cohesive material within the snap holder of the dispensing reel.

It is another aspect of the present invention to provide the elongated strip having a plurality of segments removably secured together via perforated lines of connection within the dispensing reel.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIGS. 1A and 1B show the adhesive bandage with a mobile dressing pad according to one embodiment of the present invention.

FIG. 2 shows the adhesive bandage in a dispensing reel according to one embodiment of the present invention.

### DETAILED DESCRIPTION OF THE INVENTION

Reference is made herein to the attached drawings. Like reference numerals are used throughout the drawings to depict like or similar elements of the bandage wrapping system. For the purposes of presenting a brief and clear description of the present invention, the preferred embodiment will be discussed as used for covering a wound. The figures are intended for representative purposes only and should not be considered to be limiting in any respect.

According to one embodiment, the present invention provides a cohesive bandage having a non-stick sterile pad that can be utilized to cover an open wound from contamination. The cohesive bandage can remain securely adhered to the user's skin when it is dry, oily, wet, and/or greasy. The cohesive bandage remains flush against the skin of the wearer without physically bonding to the skin via the use of adhesives. The cohesive interior and exterior layer of the bandage wrapping system include complimentary cohesive bonding layers to form a bond without attaching to the skin.

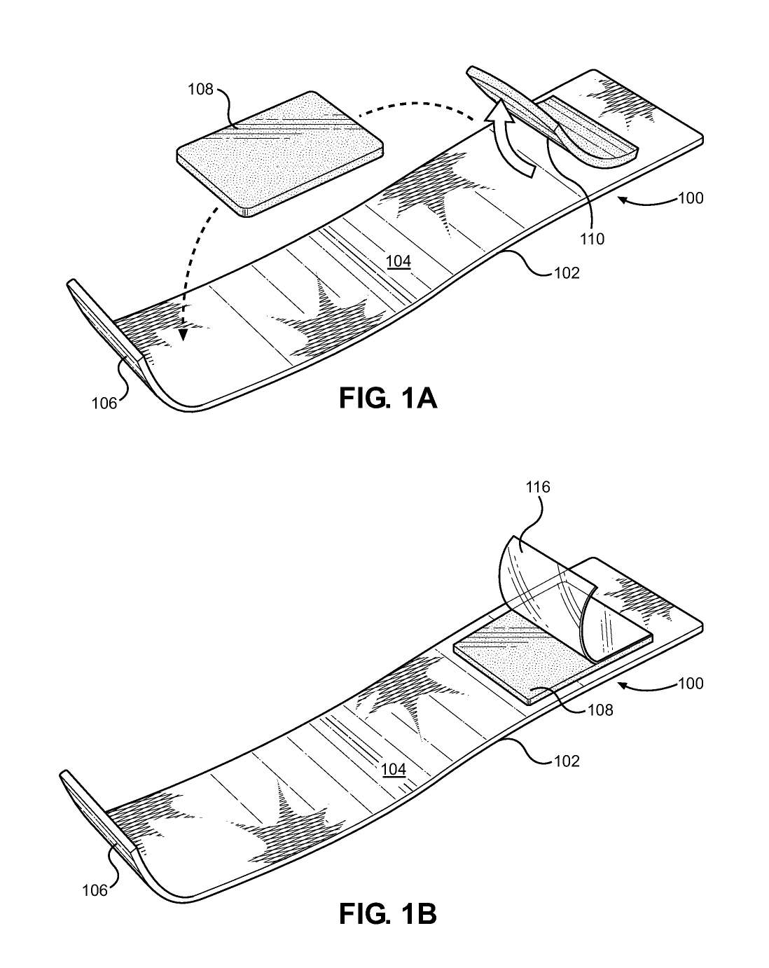

Referring now to FIGS. 1A and 1B, there is shown an adhesive bandage with a mobile dressing pad according to one embodiment of the present invention. The bandage wrapping system 100 is adapted to cover a wound on a digit or a limb. The bandage wrapping system 100 includes an elongated strip 102 of cohesive material having an interior surface 104 and an exterior surface 106. The interior surface 104 and the exterior surface 106 comprise a self-cohesive layer.

The elongated strip 102 of cohesive material can be utilized to cover a wound, scrape, or the like, that is located on the skin, without the use of adhesives. The elongated strip 102 of cohesive material includes a self-cohesive layer on the interior surface 104 and the exterior surface 106. The interior surface 104 bonds to the exterior surface 106 via the self-cohesive layer. The elongated step 102 is secured in a loop by overlapping opposing ends of the exterior layer 106 and the interior layer 104. Thus, the elongated strip 102 of cohesive material does not bond with the skin, but only with the interior surface 104 and the exterior surface 106 of the elongated strip 102 of cohesive material. When the elongated strip 102 of cohesive material is applied to a wound or scrape, the condition of the skin, for example wet and oily skin, is irrelevant because the elongated strip 102 of cohesive material does not bond with the skin. The bandage wrapping system 100 conforms to any area, for example the knuckle, by stretching and contracting with the movement of that area to stay in place.

A dressing pad 108 is configured to be variously positioned along a length of the interior surface 104 of the elongated strip 102 of cohesive material. The dressing pad 108 includes a layer of latex cohesive bonding material on a backside 110 thereof for adhering to the elongated strip 102 of cohesive material. The cohesive bonding material on the backside 110 of the dressing pad 108 may be covered by a plastic cover 116.

The dressing pad 108 adheres to the interior surface 104 of the elongated strip 102 of cohesive material via the layer of cohesive bonding material. The dressing pad 108 comprises a non-stick sterile pad. The non-stick sterile pad can be moved along the length of the elongated strip 102 of cohesive material and can be affixed to any preferred location. The cohesive bonding material on the backside 110 of the dressing pad 108 bonds with the elongated strip 102 of cohesive material affixing the dressing pad to the body of the elongated strip 102.

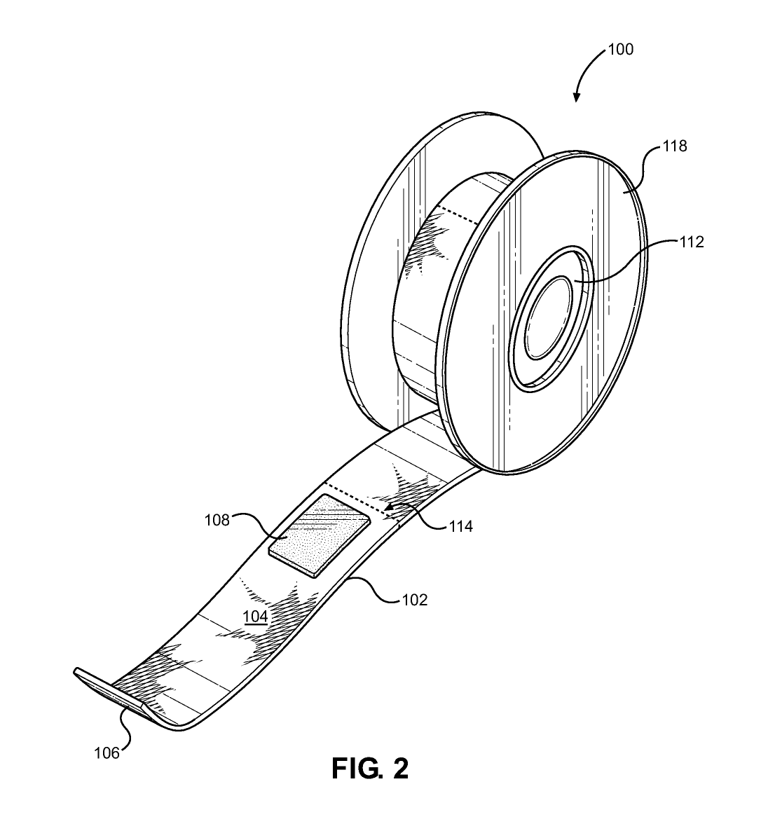

Referring now to FIG. 2, there is shown an adhesive bandage in a dispensing reel according to one embodiment of the present invention. The bandage wrapping system 100 may further include a dispensing reel 118 including a snap holder 112. The elongated strip 102 of cohesive material is secured within the holder 112 of the dispensing reel 110. The elongated strip 102 of cohesive material is in a perforated arrangement 114 within the dispensing reel 110. A strip of the bandage wrapping system 100 is secured to a user by overlapping opposing ends of the exterior layer and the interior layer.

The bandage wrapping system 100 can be dispensed from a dispensing reel 118 having a plurality of bandages of a fixed length thereon. The dispensing reel 118 can snap into a snap holder 112, thus allowing the bandage wrapping system 100 to be easily transported. The dressing pad 108 located on the interior 104 of the elongated strip 102 of cohesive material can be covered by a clear sanitary cover that can be manually pulled therefrom by a user. The bandage wrapping system 100 can be constructed from latex cohesives, foam materials, and/or other suitable materials. In a preferred embodiment, the bandage wrapping system is fabricated from a material that conforms to any area by stretching and contracting in response to movement.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A bandage wrapping system adapted to cover a wound on a digit or a limb, comprising:
an elongated strip of cohesive material having an interior surface and an exterior surface, wherein the interior surface and the exterior surface comprise a self-cohesive layer;
a dressing pad configured to be positioned along a length of the interior surface of the elongated strip of cohesive material, wherein the dressing pad includes a layer of cohesive bonding material on a backside thereof for securement to the elongated strip of cohesive material; and
wherein the dressing pad adheres to the interior surface of the elongated strip of cohesive material via the layer of cohesive bonding material;
wherein the elongated strip is secured in a loop by overlapping opposing ends of the exterior layer and the interior layer.

2. The bandage wrapping system of claim 1, further comprising a dispensing reel including a snap holder, wherein the elongated strip of cohesive material is secured within the snap holder of the dispensing reel.

3. The bandage wrapping system of claim 2, wherein the elongated strip of cohesive material is in a perforated arrangement within the dispensing reel.

4. The bandage wrapping system of claim 1, wherein the elongated strip of cohesive material comprises a latex cohesive.

5. The bandage wrapping system of claim 1, wherein the elongated strip of cohesive material comprises a foam material.

6. The bandage wrapping system of claim 1, wherein the dressing pad comprises a sterile non-stick pad.
